(19) United States
(12) Patent Application Publication
(10) Pub. No.: US 2010/0184200 A1
(43) Pub. Date: Jul. 22, 2010

(54) Cell culture device

(75) Inventors: Tsong-Rong Yan, Taipei (TW); Hung-Che Chou, Taipei (TW); Chao-Fa Lee, Taipei (TW)

Correspondence Address: BACON & THOMAS, PLLC, 625 SLATERS LANE, FOURTH FLOOR, ALEXANDRIA, VA, 22314-1176, US

(73) Assignees: Tatung University, Taipei (TW); Tatung Company, Taipei (TW)

(21) Appl. No.: 12/461,045

(22) Filed: Jul. 30, 2009

(30) Foreign Application Priority Data
Jan. 22, 2009 (TW) 098102476

(51) Int. Cl. C12M 1/34

(52) U.S. Cl. 435/287.1

(57) ABSTRACT

The present invention relates to a cell culture device which includes: a cell culture container comprising a container body and a neck connecting to the container body wherein the container contains a top wall, a bottom wall having a first opening, and at least one side wall to define a cell culture zone, and an end of the neck has a second opening for cells or culture media being injected into the cell culture zone; a biosensor chip having a detective surface which faces the top wall of the cell culture container; and a fastening component for the biosensor chip being fastened in the first opening of the bottom wall. The cell culture of the present invention can be used for real-time detection of the cell growth and further for drug screening.

### BACKGROUND OF THE INVENTION

1. Field of the Invention

The present invention relates to a cell culture device and, more particularly, to a cell culture device suitable for real-time detection of cell growth.

2. Description of Related Art

Culturing of cells has become the focus technique at the core of biotechnological industry. Cells can be cultured in vitro in an artificial way for mass production so that various kinds of biotechnological products are obtained. Therefore, cell culture techniques have been applied in industries of bulk pharmaceuticals, medicine, vaccine, bioagrochemicals, and so forth.

A purpose of the cell culture is mass production of target cells in vitro during a short period. Besides, another purpose is to figure out characteristics of different tissues in vivo by observing cell changes during culture. Effects of different drug compounds to cells can also be determined during culture, for example, occurrence of cellulotoxicity, production of cell growth promoters etc. Since different cells have different characteristics, there are various cell culture devices capable of being utilized in different applications. In combination with suitable culture media and conditions, cell culture devices designed according to characteristics of cells can promote culture efficiency in vitro.

Taiwan patent application publication No. 200801185 discloses a cell culture device, wherein a cell culture medium is imported from an inlet of a top plate, along guide holes of a guide plate, to guide channels and then to culture units of a culture plate, and finally into a collective sink.

However, such the cell culture device mainly serves to culture and collect cells so that real-time observation of cell changes can not be performed during culture. Furthermore, since this device is a large-sized system for cell culture, contamination and large consumption of culture resources may be experienced during cell culture. As a result, it is difficult for the device to be applied in drug screening.

For the purposes of prompt and accurate performance of cell culture and development of related researches, and drug screening techniques to be applicable in various drugs, there is an urgent need to develop a small-sized cell culture device with low consumption of culture resources and low possibility of contamination so as to real-timely observe cell growth in different conditions such as temperatures, humidity, CO₂ and O₂ concentrations, pH values of culture media, ionic strength, additions of target drugs etc.

### SUMMARY OF THE INVENTION

To achieve the aforesaid objectives, the present invention provides a cell culture device comprising: a cell culture container comprising a container body and a neck connecting to the container body, wherein the container comprises a top wall, a bottom wall having a first opening, and at least one side wall to define a cell culture zone, and an end of the neck has a second opening for cells or culture media being injected into the cell culture zone; a biosensor chip having a detective surface which faces the top wall of the cell culture container and is used for detection of cell growth in the cell culture zone; and a fastening component for deposition of an opposite surface to the detective surface of the biosensor chip so that the biosensor chip is received in the first opening of the bottom wall of the cell culture container.

The first opening can be arranged at any position of the bottom wall. In one aspect of the present invention, the first opening locates in the center of the bottom wall of the cell culture container.

The biosensor chip disposed in the first opening can be a sensor chip of quartz crystal microbalance (QCM). A typical structure of quartz crystal microbalance is a sandwich structure in which quartz crystal is interposed between two metal plates. Alternating voltages approximating natural resonant frequency of quartz crystal are applied on electrodes to mechanically resonate crystal. When the QCM biosensor chip is connected to a computer, the computer can analyze output signals from the detective surface of the OCM biosensor chip, and thus, states of cell growth, differentiation, and apoptosis can be recognized. As a result, the cell culture device of the present invention can real-timely detect cell growth so as to be applicable in drug screening.

In the aforesaid cell culture device, the cell culture container can further comprise a movable sealing component for sealing the second opening of the neck and, if necessary, for sealing the cell culture container. For example, the cell culture container can be a Petri dish or bottle for cell culture, and can be made of plastic or glass. In addition, the cell culture container can further comprise an inlet and an outlet disposed on the side wall. The inlet and outlet are used to connect the container to an outside fluid circulatory system. Fluid can be imported or exported through the inlet or outlet. Therefore, substitution of culture media in the cell culture container and regulation of CO₂ flux can be achieved.

Furthermore, a gasket can be fitted between the biosensor chip and the cell culture container to prevent fluid in the cell culture container from leaking outward. Hence, leakage of the cell culture media can not occur, thereby lowering possibility of cell contamination. The gasket is not limited to a specific kind of gasket as long as it can prevent leakage of the cell culture media. For instance, the gasket can be an O-ring or an O-sheet.

The fastening component can comprise an elastic cushion, on which the biosensor chip is arranged, to protect the biosensor chip. The elastic cushion is not limited to a specific kind of gasket. For example, the elastic cushion can be a rubber cushion or a silica gel cushion. Also, the kind of the fastening component is not limited as long as it can secure the biosensor chip in the first opening of the bottom wall. For example, the fastening component can further comprise two plates and a screw set. The cell culture container is secured between the plates by the screw set. Alternatively, the fastening component can further comprise two plates and a plurality of clamping members. The cell culture container is secured between the plates by the clamping members at the ends of the plates. The screw set can comprise a plurality of screw rods and a plurality of nuts. The screw rods, which extend through the plates, are fastened by the nuts at the ends of the screw rods so that the cell culture container is secured between the plates. In one aspect of the present invention, the screw rods are disposed at the terminals of the plates without penetrating the cell culture container.

The present invention also relates to a method for using the cell culture device of the present invention for drug screening.

Other objects, advantages, and novel features of the invention will become more apparent from the following detailed description when taken in conjunction with the accompanying drawings.

### BRIEF DESCRIPTION OF THE DRAWINGS

FIG. 1 shows a perspective view of a cell culture device according to Example 1 of the present invention;

FIG. 2 shows a cross-sectional view of a cell culture device according to Example 1 of the present invention;

FIG. 3 shows a perspective view of a cell culture device according to Example 2 of the present invention; and

FIG. 4 shows a perspective view of a cell culture device according to Example 3 of the present invention.

### DETAILED DESCRIPTION OF THE PREFERRED EMBODIMENT

According to the specific embodiments illustrating the practice of the present invention, a person having ordinary skill in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### EXAMPLE 1

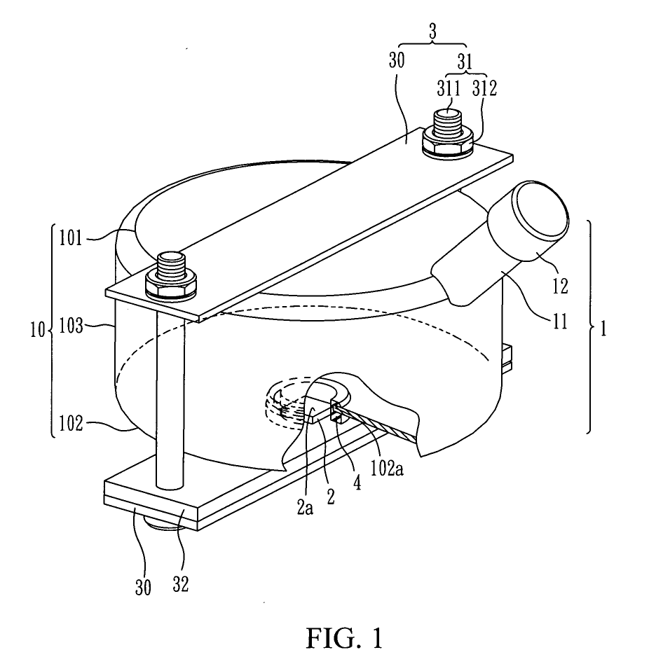

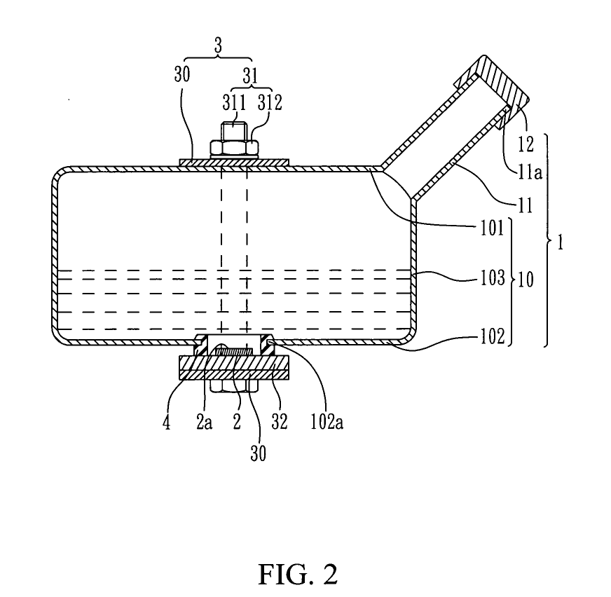

Referring to FIGS. 1 and 2, a perspective view and a cross-sectional view of a cell culture device in the present example are respectively shown. The cell culture device of the present invention mainly includes: a cell culture container 1, a biosensor chip 2, and a fastening component 3.

The cell culture container 1 comprises a container body 10, a neck 11, and a movable sealing component 12. One end of the neck 11 connects the container body 10, and the other end thereof has a second opening 11a used as an entrance for importing cells or cell culture media into a cell culture zone Z. The sealing component 12 is connected to the end of the neck 11 having the second opening 11a. Therefore, the sealing component 12 can seal the cell culture container 1, if necessary. The aforesaid container body 11 comprises a top wall 101, a bottom wall 102, and at least one side wall 103, thereby defining the cell culture zone Z. The bottom wall 102 has a first opening 102a in the center thereof to receive the biosensor chip 2. However, the location of the first opening 102a is not limited to the center of the bottom wall 102. Besides, the cell culture container 1 used in the present invention is not limited. In the present example, a glass box for cell culture can be used as the cell culture container 1.

The biosensor chip 2 has a detective surface 2a which faces the top wall 101 of the cell culture container 1 and it is used to detect the cell growth, differentiation, and apoptosis in the cell culture zone Z. In the present example, the used biosensor chip 2 is a QCM chip.

The fastening component 3 is mainly used for deposition of an opposite surface to the detective surface 102a of the biosensor chip 2, and hence, the biosensor chip 2 can be secured in the first opening 102a of the bottom wall 102 of the cell culture container 1. In general, the fastening component 3 can comprise an elastic cushion 32. The elastic cushion 32 serves for deposition of the opposite surface to the detective surface 102a of the biosensor chip 2. Therefore, the elastic cushion 32 functions as to protect the biosensor chip 2. Materials of the elastic cushion 32 are not limited, and for example, it can be rubber or silica gel. In the present example, the fastening component 3 further includes two plates 30 and a screw set 31. Thus, the cell culture container 1 is secured between the two plates 30 by the screw set 31. In addition, the elastic cushion 32 is arranged between the plates 30 and the cell culture container 1. The foregoing screw set 31 includes a plurality of screw rods 311 and nuts 312. The plates 30 are penetrated by the screw rods 311 at the two ends of the plates 30. Besides, the nuts 312 are threadably engaged at the two ends of the screw rods 311 so that the cell culture container 1 is interposed between the plates 30.

A gasket 4 can be disposed between the biosensor chip 2 and the cell culture container 1 so as to prevent fluid in the cell culture container 1 from leaking outward. In the present example, this gasket 4 is an O-ring. This O-ring has a groove along the outer surface, and the edge of the first opening 102a in the bottom wall 102 of the cell culture container 1 can be received in the groove of the O-ring. Hence, fluid leakage can not occur from the first opening 102a.

The biosensor chip 2 of the present invention can be connected to a computer analysis system (not shown). As a result, signals output from the biosensor chip 2 can be real-timely analyzed, and thus, states of cell growth in the cell culture zone Z can be recognized to achieve the purpose of real-timely monitoring cells.

### EXAMPLE 2

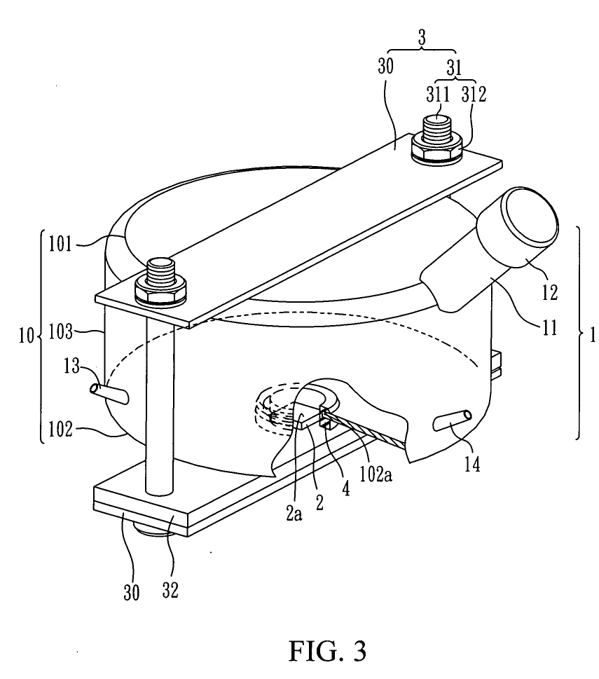

With reference to FIG. 3, there is shown a perspective view of a cell culture device in the present example. The cell culture device of the present example is substantially identical to that of Example 1 except that the cell culture container 1 further comprises an outlet 13 and an inlet 14 arranged on the side wall 103. The outlet 13 and the inlet 14 can connect the cell culture container 1 to an outside fluid circulatory system. Hence, fluid can be imported from or exported outside the cell culture container 1. It is advantageous to replace culture media in the cell culture zone Z and also to regulate CO₂ flux.

### EXAMPLE 3

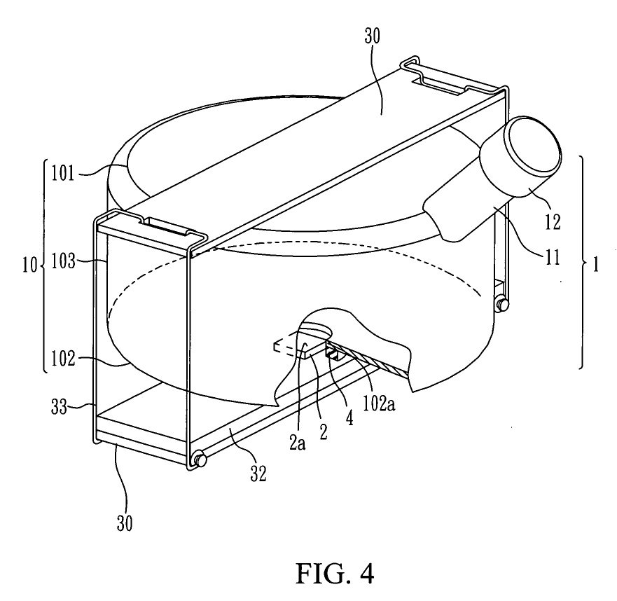

With reference to FIG. 4, there is shown a perspective view of a cell culture device in the present example. The cell culture device of the present example is substantially identical to that of Example 1 except that the fastening component 3 further comprises two plates 30 and plural clamping members 33 in addition to the elastic cushion 32. The clamping members 33 are placed at the two ends of the plates 30. Besides, the plates 30 can have grooves or holes to allow the terminal ends of the clamping member 33 to be received therein. Thus, the cell culture container 1 can be disposed between the plates 30. In the present example, an O-sheet is used as the gasket 4.

In conclusion, the cell culture device of the present invention can be connected to an outside computer monitoring system. Then, signals output from the biosensor chip can be analyzed so as to recognize states of cells in the cell culture zone. Hence, the cell culture of the present invention can be used for prompt and accurate cell culture and related researches, and also can be applied in cell screening for different drugs.

Although the present invention has been explained in relation to its preferred embodiment, it is to be understood that many other possible modifications and variations can be made without departing from the scope of the invention as hereinafter claimed.

What is claimed is:

1. A cell culture device comprising:
a cell culture container comprising a container body and a neck connecting to the container body, wherein the container comprises a top wall, a bottom wall having a first opening, and at least one side wall to define a cell culture zone, and an end of the neck has a second opening for cells or culture media being injected into the cell culture zone;
a biosensor chip having a detective surface which faces the top wall of the cell culture container and is used for detection of cell growth in the cell culture zone; and
a fastening component for deposition of an opposite surface to the detective surface of the biosensor chip so that the biosensor chip is received in the first opening of the bottom wall of the cell culture container.

2. The cell culture device as claimed in claim 1, wherein the cell culture container further comprises a movable sealing component for sealing the second opening of the neck.

3. The cell culture device as claimed in claim 1, wherein the first opening locates in the center of the bottom wall.

4. The cell culture device as claimed in claim 1, further comprising a gasket between the biosensor chip and the cell culture container, to prevent fluid in the cell culture container from leaking outward.

5. The cell culture device as claimed in claim 4, wherein the gasket is an O-ring or an O-sheet.

6. The cell culture device as claimed in claim 1, wherein the biosensor chip is a sensor chip of quartz crystal microbalance.

7. The cell culture device as claimed in claim 1, wherein the cell culture container is a cell culture dish or a cell culture bottle.

8. The cell culture device as claimed in claim 1, wherein the cell culture container is made of plastic or glass.

9. The cell culture device as claimed in claim 1, wherein the cell culture container further comprise an inlet and an outlet disposed on the side wall for importing or exporting fluid in or out of the cell culture container.

10. The cell culture device as claimed in claim 1, wherein the fastening component comprises an elastic cushion, on which the biosensor chip is arranged, to protect the biosensor chip.

11. The cell culture device as claimed in claim 10, wherein the elastic cushion is a rubber cushion or a silica gel cushion.

12. The cell culture device as claimed in claim 10, wherein the fastening component further comprising two plates and a screw set, and the cell culture container is secured between the plates by the screw set, wherein the elastic cushion is disposed between the plates and the cell culture container.

13. The cell culture device as claimed in claim 12, wherein the screw set comprises a plurality of screw rods and a plurality of nuts, and the screw rods, which extend through the plates, are fastened by the nuts at the ends of the screw rods so that the cell culture container is secured between the plates.

14. The cell culture device as claimed in claim 13, wherein the screw rods are disposed at the terminals of the plates.

15. The cell culture device as claimed in claim 10, wherein the fastening component further comprises two plates and a plurality of clamping members, and the cell culture container is secured between the plates by the clamping members at the ends of the plates, wherein the elastic cushion is disposed between the plates and the cell culture container.

16. A method for using the cell culture device as claimed in any one of claims 1 to 15 for drug screening.
